(19) United States
(12) Patent Application Publication
(10) Pub. No.: US 2026/0157785 A1
(43) Pub. Date: Jun. 11, 2026

(54) ELECTROSURGICAL INSTRUMENT

(71) Applicant: Erbe Elektromedizin GmbH, Tuebingen (DE)

(72) Inventor: Tanja HEIDER, Kirchentellinsfurt (DE)

(21) Appl. No.: 19/407,018

(22) Filed: Dec. 3, 2025

(30) Foreign Application Priority Data
Dec. 6, 2024 (EP) 24218038.8

(51) Int. Cl. A61B 18/08; A61B 18/00

(52) U.S. Cl. CPC A61B 18/085; A61B 2018/00107; A61B 2018/00601; A61B 2018/0063

(57) ABSTRACT

An electrosurgical instrument configured for thermal cutting of biological tissue of a patient is disclosed. The instrument comprises at least one movable jaw and at least one jaw including a thermal cutting element with an electrically conductive cutting conductor and an electrically conductive return conductor that are electrically connected with each other. The cutting element is arranged so that the cutting conductor is at least thermally uncovered on a side of one jaw facing another jaw and so that the return conductor is surrounded by an insulating body. The return conductor comprises a lower electrical resistance in a current flow direction and a higher thermal conductivity than the cutting conductor. In doing so, during supply of current to the cutting element at the return conductor a considerably lower amount of heat is created than in the cutting conductor and the return conductor can be used for heat dissipation.

### CROSS REFERENCE TO RELATED APPLICATION

This application claims the benefit of European Patent Application No. 24218038.8, filed Dec. 6, 2024, which is incorporated herein by reference in its entirety.

### TECHNICAL FIELD

The invention refers to an electrosurgical instrument, particularly for thermal cutting of biological tissue of a human or animal patient, as well as for achieving additional tissue effects where appropriate.

### BACKGROUND

Electrosurgical instruments with thermal cutting elements are known from the prior art in different embodiments.

WO 2023/187737 A1 discloses a surgical instrument having a thermal cutting device. The thermal cutting device comprises a longitudinal support with a proximal and a distal end and a cutting edge, that is arranged along the upper surface thereof. Moreover, the cutting device comprises a dielectric insulator that is arranged along at least one side of the substrate and at least partly extends along the latter from the proximal to the distal end. Additionally, the cutting device comprises at least one resistance element that is suitable for connection with an energy source and is arranged in thermal connection with the substrate. The at least one resistance element is thereby arranged, so that it extends along the dielectric insulator to a distal end section thereof. Moreover, the cutting device comprises an encapsulation material that is arranged on the dielectric insulator as well as on the at least one resistance element. The distal end of the substrate comprises a mechanical interface that is configured to get into engagement with a section of the jaw in order to attach the substrate to the jaw.

Other thermal cutting elements are described in WO 2023/187735 A1, US 2023/0363812 A1, WO 2023/187736 A1, US 2023/0310063 A1, EP 3 861 950 A1, EP 3 769 709 B1, US 2022/0378494 A1 and US 2023/0285064 A1.

In US 2023/240740 A1 a thermal cutting element is described in which heating elements are applied on an electrically insulating support. The heating elements can have a varying cross-section area along the length. In addition, the heating elements comprise different sections that can be configured, for example, in linear or meandering manner in order to create a desired temperature profile.

A method for producing a thermal cutting element for a surgical instrument is described in EP 4 076 239 B1. In the method at least on a section of a support a coating is applied by means of a plasma electrolytic oxidation method. Then a heating element is applied onto this coating.

### SUMMARY

All heating elements that can be found in the prior art comprise support materials having a comparably high thermal conductivity, so that the entire jaw can heat up also at undesired positions, such as the outer side of the jaw. This can make the surgical operation more difficult for the surgeon, because the instrument jaw—particularly during longer surgical operations—can thermally stick to the tissue of the patient at undesired positions in the operating field and can unintentionally damage the tissue there, which can be accompanied with an extended convalescence of the patient after the surgical operation.

Starting therefrom, one object of the present invention is to provide an improved electrosurgical instrument for thermal cutting of biological tissue. In the ideal case, the jaws of the instrument shall preferably not heat up at all or only to a minor extent during use.

This object is solved by means of an electrosurgical instrument as described herein.

The surgical instrument according to the invention is particularly configured for thermal cutting of biological tissue of a patient. The instrument comprises at least two jaws, wherein at least one of the jaws is configured to be moved relative to the other jaw toward the latter or away from the latter. For example, one of the jaws is configured to be pivoted around a pivot axis by means of a pivot device. Also both jaws can be movably configured toward and away from each other in forceps-like manner. At least one of the jaws comprises a thermal cutting element having an electrically conductive cutting conductor and an electrically conductive return conductor. The cutting element is arranged, so that the cutting conductor is at least thermally uncovered on a side of the jaw facing the other jaw and so that the return conductor is (entirely) enclosed by an insulating body. The cutting conductor can be configured in metallic bare manner or covered with a thermally conductive, electrically insulating coating. For example, the coating can have hydrophobic characteristics, whereby sticking of the cutting conductor to the biological tissue of the patient can be avoided.

The cutting conductor and the return conductor can be connectable to a current source and can be supplied from the latter. Such a current source can be a direct current source, but also an alternating current source, for example a high frequency source (HF source).

A particularity of the present invention is that the return conductor comprises a lower electrical resistance and preferably a higher thermal conductivity than the cutting conductor. The insulating body is configured in electrically and thermally insulating manner, so that during supply of the cutting element with current, heat is predominantly produced on the cutting conductor and is there impeded to be dissipated in backward direction, particularly by the insulating body. For example, the insulating body can consist of silicone. During supply of the cutting element with current, considerably less heat can be produced in the return conductor than in the cutting conductor, because the return conductor has a lower electrical resistance than the cutting conductor. Particularly, the return conductor comprises a core (the return conductor core) that is covered by a coating. Due to the coating the characteristics of the return conductor can be influenced, particularly the electrical resistance and the thermal conductivity of the return conductor. Preferably, the electrical resistance of the return conductor can be configured to be lower due to the coating than the electrical resistance of the return conductor core. Preferably, the return conductor core consists of the same material as the cutting conductor.

The cutting conductor and the return conductor are electrically and physically connected with each other. Particularly in conductor direction—that means in and opposite to the direction in which the current flows during use of the cutting element—the return conductor comprises a higher thermal conductivity than the cutting conductor. The coating of the return conductor particularly comprises a higher thermal conductivity than the basic material of the return conductor core. The coating provides that the electrical resistance of the return conductor, that means the entire composition of the return conductor core and the coating, is lower than the electrical resistance of the cutting conductor. In addition, due to the coating the return conductor comprises a higher thermal conductivity than the return conductor core without coating. Because of the high thermal conductivity of the return conductor, the heat created at the return conductor can be dissipated, which is particularly advantageous if the return conductor has a particularly small cross-section. In doing so, it can be avoided that the return conductor (total composition of return conductor core and coating) is overheated. In this manner, the jaw can be configured particularly slim and particularly flat without locally transporting too much heat from the return conductor to the support part of the jaw and thus to the outer side of the jaw during cutting.

The cutting conductor and the return conductor are preferably one monolithic body. The latter is, for example, at least substantially configured in U-shaped manner, wherein the cutting conductor and the return conductor can form one leg of the U-shaped cutting element in each case.

The jaw particularly comprises an oblong support part that extends from a proximal region, in which a hinge device for pivotable support of the other jaw can be arranged, to a distal end region. The support part can limit a holding space in which the cutting element is preferably vertically arranged. Thereby the cutting conductor is arranged above the return conductor.

The cutting conductor and the return conductor consist preferably of one and the same basic material. On the latter different coatings can be provided at different locations; individual locations, for example the cutting conductor, connection sections or the like, can also be free from coatings.

The U-shaped cutting element is preferably open toward the proximal area of the jaw. At the proximal end, connection sections or connection elements can be electrically connected to the cutting conductor and the return conductor, by means of which the cutting element can be connected with supply lines. Via the latter, the cutting element can be connected with a current source and can be supplied therefrom. Due to the monolithic configuration of the cutting element, particularly at the distal end area of the jaw, in which the cutting conductor is physically connected to the return conductor—that means electrically and thermally connected—no connection seam, for example welding seam or brazing seam, is necessary. The risk of conductor breakages between the cutting conductor and the return conductor is significantly reduced during use of the instrument and the mechanical stress of the cutting element related therewith.

The return conductor core is preferably at least partly provided with a coating extending in longitudinal direction. This coating preferably covers the entire outer circumference of the return conductor core. The coating allows to manufacture the return conductor core and the cutting conductor from one material as well as monolithically, whereby the requirement of an additional element for connecting the two conductors (cutting and return conductor) is omitted. It is preferred that the entire return conductor is provided with the coating, whereby the heat produced at the return conductor can be dissipated therefrom extensively by means of the coating.

The coating consists preferably from a material having a lower specific electrical resistance than the basic material of the cutting conductor. In addition, the material of the coating preferably comprises a higher specific thermal conductivity than the basic material of the cutting conductor. The cutting conductor and the return conductor can consist of the same material, such as stainless steel, of a nickel-base alloy that can comprise iron, molybdenum, niobium, cobalt, manganese, copper, aluminum, titanium, silicon, carbon, sulfur, phosphorus and boron or of an iron-chromium-aluminum alloy. The coating consists, for example, of copper, silver, and/or aluminum. The specific thermal conductivity of the coating is particularly higher than 200 W/(m·K).

Particularly the cutting conductor comprises a larger cross-section area and a higher specific resistance than the return conductor, whereby the electrical resistance of the cutting conductor is higher, preferably considerably higher than the electrical resistance of the return conductor, provided with the coating having lower electrical resistance and higher thermal conductivity. Thereby the heat created in the cutting element can be mainly produced at the cutting conductor, wherein the return conductor remains comparably cold and serves for heat dissipation.

In an edge area of the jaws, they preferably comprise at least one sealing electrode respectively, which is arranged with distance to the cutting conductor. Particularly, the cutting conductor is surrounded at least at its lateral flanks between the distal and the proximal end of the jaw by the sealing electrode.

The sealing electrodes are particularly configured to be connected to a current source, for example an HF current source. The sealing electrodes and the cutting element can be supplied by means of one (single) HF current source.

The sealing electrodes and the cutting conductor can be supplied from a common supply source, for example HF current source. A network, for example a transformer, provided in the instrument can thereby be used to provide the different required voltages from the voltage of the supply source, which are required for the operation of the sealing electrodes and the cutting element. The network (the transformer) can be part of the instrument or part of the supplying generator. It is, however, also possible to supply the sealing electrodes and the cutting element with respectively own (separate) HF current sources.

Particularly also the other (second) jaw comprises at least one sealing electrode in an edge region. The sealing electrode is preferably identically configured compared with the sealing electrode of the one (first) jaw, so that both are arranged on top of each other, but with distance to one another with the jaws being closed. The two sealing electrodes are connected to the poles of a supplying generator. In the second jaw an elastic counter-pressing body is arranged against which the cutting conductor runs during closing of the jaws in order to slightly deform the counter-pressing body elastically.

Preferably, the insulating body (and also the counter-pressing body) consists of a material having a lower thermal conductivity than the cutting conductor, whereby the cutting conductor can be largely thermally insulated from the return conductor. The heat produced by the cutting conductor can thus be concentrated to a narrow strip of the biological material and can become effective there.

On the cutting conductor at least one attachment projection can be configured, which is surrounded by the insulating body. Preferably the attachment projection comprises at least one undercut section. The attachment projection is particularly entirely surrounded by the insulating body, whereby it can be avoided that the cutting conductor mechanically detaches from the insulating body or can be pulled out of the insulating body, for example if the cutting conductor sticks to the biological tissue and the instrument is further moved.

At least one distance element can be arranged between the cutting conductor and the return conductor. The distance element can consist, for example, of an electrically and thermally insulating material, for example ceramic, such as ZrO₂ or AlO₂. The material from which the distance element consists can particularly withstand temperatures of more than 350° C.

In an embodiment the distance element can have a (planar) upper side and a (planar) lower side, for example. Preferably, the distance element abuts with its upper side against the cutting conductor and/or with its lower side against the return conductor. In doing so it can be avoided that the cutting conductor bends in case of mechanical stress. The insulating body comprises a certain elastic deformability, particularly if it consists of silicone. Due to the distance element the cutting conductor can be supported on the return conductor, whereby a deformation of the cutting conductor can be avoided.

In another embodiment multiple distance elements, particularly at least three distance elements, are arranged between the cutting conductor and the return conductor. Each of the distance elements can comprise a return conductor cavity, for example on its lower side, inside which the return conductor is placed, so that the latter is at least partly surrounded and held by the distance element, whereby the distance elements contribute to position and fixate the cutting element consisting of cutting conductor and return conductor in the one jaw. Preferably, the return conductor cavity is open at one lateral side. This simplifies the assembly of the cutting element on the jaw. Preferably the multiple distance elements, particularly at least three distance elements, are arranged in a manner, so that the side alternates toward which the return conductor cavities are opened.

Preferably the multiple distance elements have a support surface on which the cutting conductor is supported. Particularly the lower side of the cutting conductor is supported on the support surface. The distance element comprises at least one lateral stop projecting from the support surface and against which the cutting conductor abuts laterally. Preferably the multiple distance elements are arranged so that the lateral side alternates at which the at least one stop projects from the support surface.

It is preferred if the other (second) jaw comprises the counter-pressing body that is arranged on a side facing the one (first) jaw and there defines a (planar) counter-pressing surface for the cutting conductor of the one (first) jaw. The counter-pressing body is preferably configured in electrically and thermally insulating manner. The counter-pressing body preferably comprises a certain elasticity so that the counter-pressing body can slightly deform if parts of the cutting element penetrate therein. Due to the elasticity the counter-pressing body provides that the tissue abuts against the cutting conductor during cutting. The cutting conductor particularly projects from the surface formed by the insulating body in the direction toward the other jaw. Preferably the cutting conductor can project about 0.10 mm, 0.15 mm or more.

Particularly the counter-pressing body can be configured, so that the cutting conductor can penetrate therein along the (entire) cutting conductor, preferably with uniform depth, when the jaws are closed.

### BRIEF DESCRIPTION OF THE DRAWINGS

Further details of advantageous embodiments of the invention are derived from the dependent claims, the drawing or from the description. The drawings show:

FIG. 1 an example of an electrosurgical instrument according to the invention in a perspective view;

FIG. 2 an example for the tool of the instrument at the distal end of the instrument in a detailed illustration;

FIG. 3 an example of the first jaw of the instrument in a top view;

FIG. 4 an example for the second jaw of the instrument in a top view;

FIG. 5 an example for the cutting element with a part of the insulating body in a detailed illustration;

FIG. 6 an example for the two jaws of the instrument in a closed condition in a longitudinal cut illustration;

FIG. 7 an example for the two jaws of the instrument in closed condition in a cross-section illustration;

FIG. 8 another example for the two jaws of the instrument in closed condition in a cross-section illustration;

FIG. 9 another example of the first jaw of the instrument in a longitudinal cut illustration;

FIG. 10 a detailed illustration of the cutting element with the distance elements with view from one side;

FIG. 11 a detailed illustration of the cutting element with the distance elements with view from another side; as well as

FIG. 12 the lower jaw of the instrument in a cross-section illustration.

### DETAILED DESCRIPTION

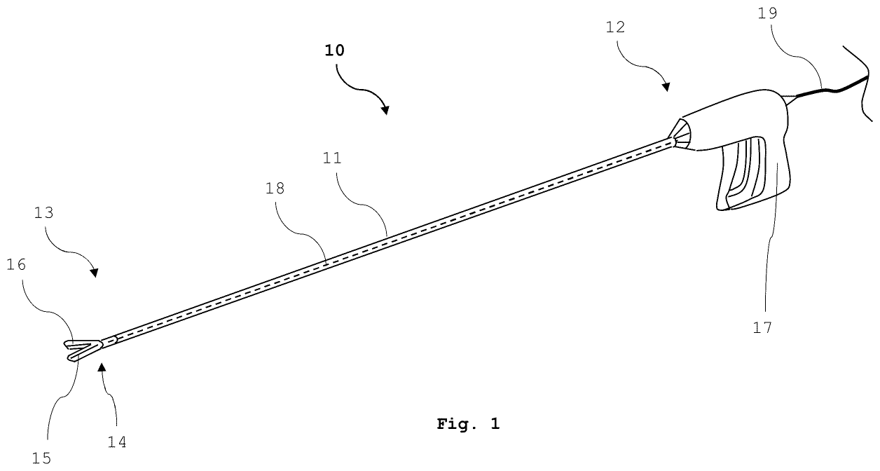

FIG. 1 shows an example of the electrosurgical instrument 10 that is configured for thermal cutting of biological tissue of a patient. The instrument 10 comprises a longitudinal shank 11, which extends from a proximal end 12 of the instrument 10 to a distal end 13 of the instrument 10. At the distal end 13 of the electrosurgical instrument 10 a tool 14 is arranged, which comprises a first jaw 15 and a second jaw 16, whereby at least one of the two jaws 15, 16, can be moved relative to the other.

On the proximal end 12 of the electrosurgical instrument 10 a handle 17 is arranged for operating the tool 14. In the shank 11 channels can be provided in which electrical supply lines 18 can be arranged extending from the proximal end 12 to the distal end 13 of the instrument 10, in order to electrically supply the tool 14 having cutting and sealing electrodes arranged therein. In addition, in the shank 11 one or more control wires can be provided of which at least one is connected with at least one of the two jaws 15, 16, in order to open and close the latter. On the handle 17 of instrument 10 an instrument supply line 19 is attached by means of which the electrosurgical instrument 10 can be connected to a supply apparatus for supply of the instrument 10 with voltage, current or other media.

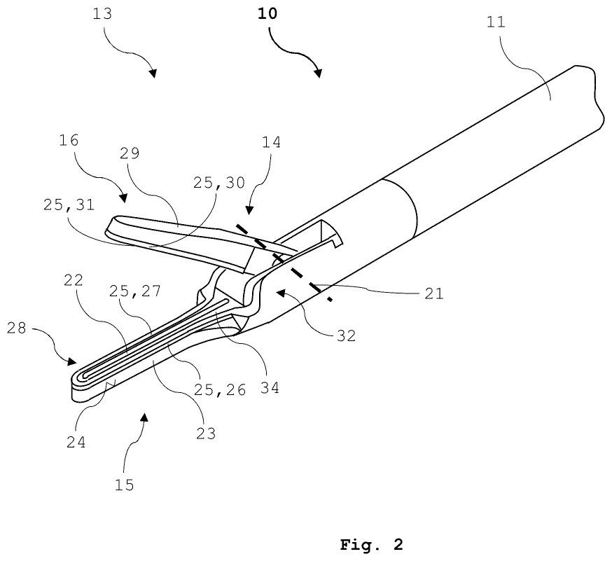

FIG. 2 shows a detailed illustration of the tool 14 that is arranged on the distal end 13 of shank 11. The two jaws 15, 16 can be opened and closed in the manner of a forceps. For this purpose at least one of the two jaws 15, 16—in the present embodiment the second jaw 16—is pivotable by means of a hinge device indicated by its pivot axis 21 relative to the first jaw 15, which is immovably arranged. Alternatively, also both jaws 15, 16 can be pivotably arranged toward and away from each other. The hinge device can be realized by means of one or two pivot bearings, a slotted guide, a spring hinge or the like.

In at least one of the two jaws 15, 16 a thermal cutting element 22 is arranged for thermal cutting of biological tissue. In FIG. 2 the jaws 15, 16 are configured in a particularly narrow and slim manner and are configured at least substantially straight. Different to the illustration in FIG. 2 the jaws 15, 16 can also have a slight curvature, whereby the electrosurgical instrument allows the preparation of organs or other biological tissues, for example. The electrosurgical instrument 10 serves particularly for cutting, separating, closing and sealing of vessels, for example blood vessels.

The first jaw 15 is formed by a rigid support part 23 consisting, for example, of metal that can have an electrical insulation at its outer side 24. Alternatively, the support part 23 can also partly or entirely consist of a mechanically stable, less flexible or non-flexible and electrically insulating plastic. Also, the support part 23 can be a composite part and made, for example, of a metal inlay overmolded with a plastic.

Along its two edge regions 25 the support part 23 of first jaw 15 is provided with sealing electrodes 26 and 27, that can be connected by means of non-illustrated lines with an electrical generator. The two sealing electrodes 26 and 27 can be physically and electrically connected with each other in a distal end section 28 of first jaw 15, as depicted in FIG. 2. Alternatively, however, also separate sealing electrodes 26 and 27 can be provided that are at equal or different electrical potentials and are physically not connected in the distal end section 28.

The second jaw 16 also comprises a support part 29 that can be again made of metal or also a plastic or a metal-plastic composite part. At its outer edge support part 29 supports the sealing electrodes 30, 31 that extend from an area 32 close to the joint up to the distal end section 28. The contour of the support part 29 of second jaw 16 is congruent to the contour of the first jaw 15. If both jaws are closed the sealing electrode 26 is aligned with the sealing electrode 30. In addition, the sealing electrode 27 is aligned with sealing electrode 31. Moreover, the sealing electrodes 30, 31 can be connected electrically and physically with each other in a distal end section 28 of second jaw 16.

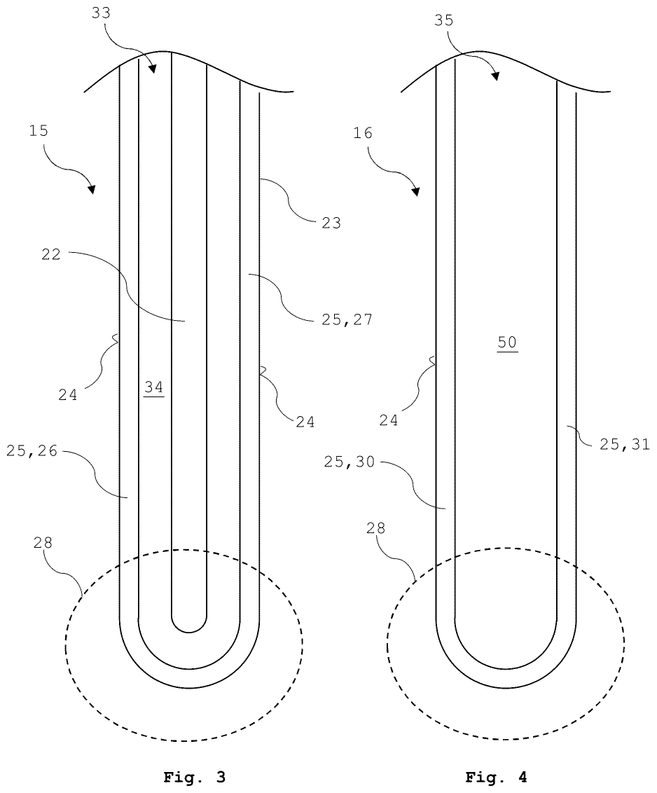

FIG. 3 shows a top view onto first jaw 15. The support part 23 of first jaw 15 comprises a groove 33 in which cutting element 22 is located. The cutting element 22 is surrounded by an insulating body 34, so that the top side of cutting element 22, where cutting contactor 36 is arranged, projects from insulating body 34.

FIG. 4 shows a top view of second jaw 16. In this embodiment no cutting element 22 is arranged in second jaw 16. Alternatively, a cutting element 22 can also be arranged in second jaw 16, just as in the first jaw 15. In the example illustrated in FIG. 4, support part 29 of second jaw 16 comprises a groove 35 in which a counter-pressing body 50 is arranged.

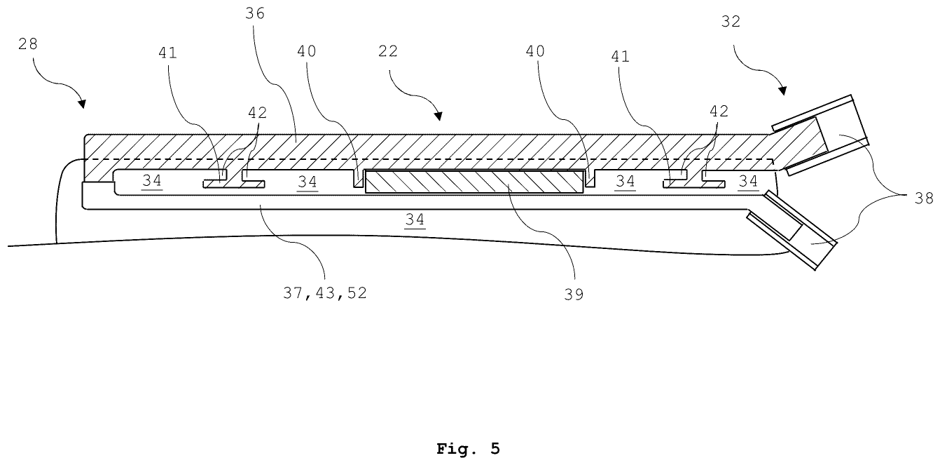

FIG. 5 depicts the cutting element 22 in longitudinally cut illustration. The cutting element 22 comprises a cutting conductor 36 and a return conductor 37. The cutting conductor 36 extends from the area 32 close to the joint up to the distal end section 28. In the area 32 close to the joint the cutting conductor 36 and the return conductor 37 can be electrically contacted, for example in a form- and/or friction-fit manner. For example, the cutting conductor 36 and the return conductor 37 can be contacted by means of a silver wire without requiring additional connection elements. In the example shown in FIG. 5 a connection element 38 is attached to the cutting conductor 36. The connection element 38 can be a connection sleeve, for example a crimping sleeve or the like. The return conductor 37 extends from the distal end section 28 up to the area 32 close to the joint.

The return conductor 37 is arranged below the cutting element 36. In the distal end section 28 the cutting conductor 36 and the return conductor 37 are electrically and physically connected. In the area 32 close to the joint the return conductor 37 also comprises a connection element 38 in the example shown in FIG. 5. Via the connection elements 38 cutting conductor 36 and return conductor 37 can be connected to a current source.

The current source can be the same current source that is also used for the sealing electrodes, wherein an adaption network can be arranged between the current source and the cutting element. The adaption network can be configured to distribute the electrical power provided by the current source on the sealing electrodes and the cutting element and to adapt the supplied voltage to the required voltage. The same applies to the supplied current and the required current. The cutting element 22 can, however, also be connected to a separate current source.

A distance element 39 is arranged between cutting conductor 36 and return conductor 37, wherein the distance element 39 consists of a thermally insulating and electrically insulating material. The distance element 39 is configured at least substantially planar and abuts with its top side against the lower side of the cutting conductor 36 and with its lower side against the top side of the return conductor 37. The distance element 39 serves to avoid that the cutting conductor 36 bends in case of mechanical stress, but is instead supported via the distance element 39 on the return conductor 37. The distance element 39 is arranged between two positioning projections 40 that project on the lower side of the cutting conductor 36 in direction toward the return conductor 37, however do not touch the latter. According to FIG. 5, on the side of the cutting conductor 36 facing the return conductor 37 in addition two attachment projections 41 are arranged that project from the cutting conductor 36 in direction toward the return conductor 37. The attachment projections 41 comprise undercut sections 42.

The return conductor 37 comprises a core 52, which is surrounded by a coating 43, so that the return conductor 37 has a lower electrical resistance than the cutting conductor 36. In addition, the return conductor 37 has a larger thermal conductivity in current flow direction than the cutting conductor 36.

The coating 43 covers the core 52 of return conductor 37 circumferentially preferably completely. In the example shown in FIG. 5 the coating 43 extends from the distal end section 28 up to the area 32 close to the joint. Different to the illustration in FIG. 5 the coating 43 can also be present only in a section of core 52 of return conductor 37, preferably adjoining the distal end section 28, where the cutting conductor 36 is electrically and physically connected with return conductor 37. The return conductor 37 and also a part of the cutting conductor 36 is preferably embedded in the insulating body 34, so that only an upper part of the cutting conductor 36 protrudes from insulating body 34.

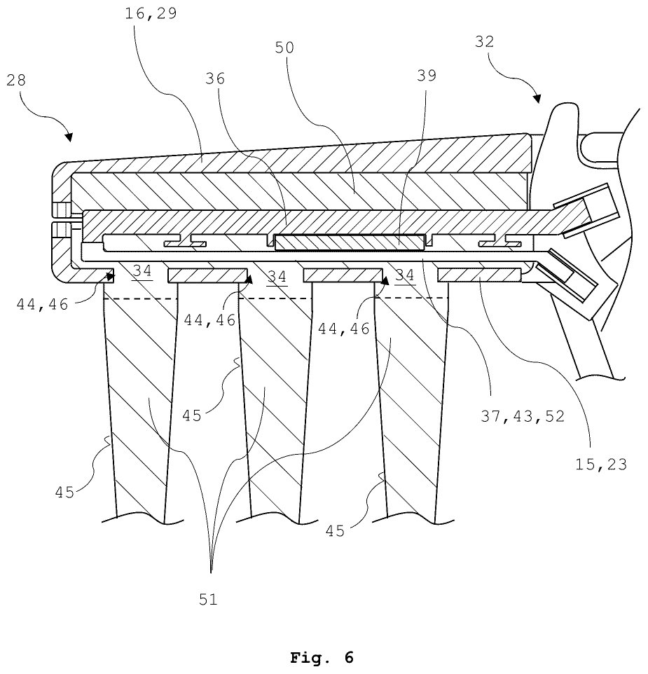

In FIG. 6 the tool 14 is illustrated longitudinally cut with view from the side. The cutting element 22 is embedded in the insulating body 34, so that the return conductor 37 is entirely surrounded by insulating body 34. The insulating body 34 comprises at its lower side multiple insulating body feet 51, which comprise one undercut section 44 respectively. The insulating body feet 51 are arranged at positions, where openings 46 are provided in the support part 23 of first jaw 15.

For the assembly of cutting element 22, it is overmolded with insulating body 34. The insulating body feet 51 additionally comprise a tapering section 45 that can serve to pull the insulating body 34 at the insulating body feet 51 through the openings 46 in the support part 23 of first jaw 15 until the undercut section 44 is seated inside the opening 46. Due to the undercut section 44 the insulating body 34 including cutting element 22 is seated in form-fit manner in the support part 23 of first jaw 15. After insertion of insulating body 34 in support part 23 the insulating body feet 51 can be cut away.

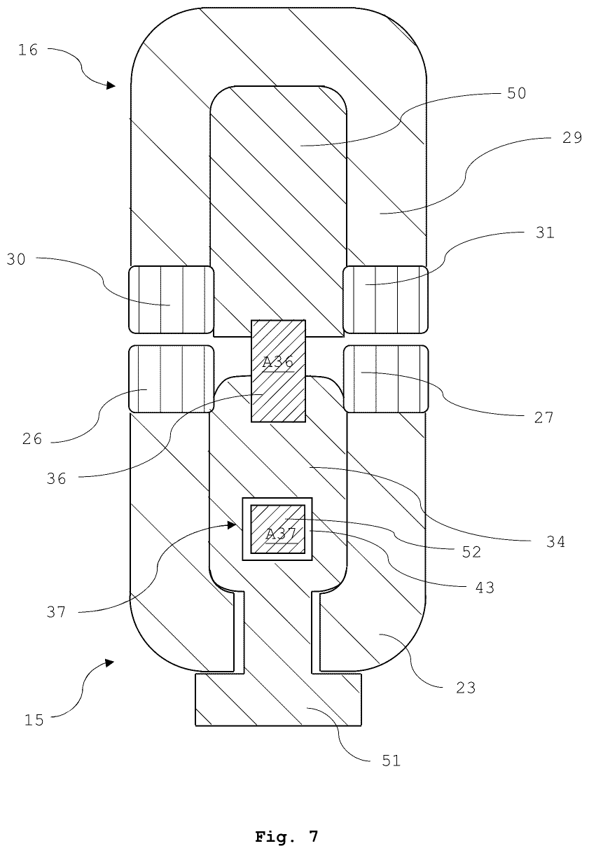

FIG. 7 shows a cross-section through the tool 14 of instrument 10. Insofar the above explanations apply with reference to the already introduced reference signs. The tool 14 is shown with the jaws 15, 16 being closed. The cutting conductor 36 overlaps in the closed condition of the jaws 15, 16 at least partly with counter-pressing body 50. The counter-pressing body 50 serves to guarantee that the tissue of the patient that is to be cut is in contact with cutting conductor 36 in order to allow a clean cut.

Moreover, the cross-section area A36 of cutting conductor 36 is larger than the cross-section area A37 of return conductor 37. In the embodiment depicted in FIG. 7 the cross-section area A37 of return conductor 37 is approximately half of the cross-section area A36 of cutting conductor 36. In the example shown in FIG. 7 the return conductor 37 is in addition coated along its entire circumference with a coating 43. The small cross-section area A37 of return conductor 37 first results in that the electrical resistance of return conductor 37 without the coating is higher than that of the cutting conductor. Because the coating 43 consists, however, of particularly low-ohmic material, the electrical resistance of return conductor 37 is smaller than the electrical resistance of cutting conductor 37 even in spite of its smaller cross-section area A37. For example, a width of the core 52 of the return conductor has an amount of between 0.2 and 0.5 mm and a height has an amount of between 0.2 and 0.5 mm. The coating thickness of coating 43 has an amount of between 0.02 mm and 0.08 mm. The width of the cutting conductor 36 also has an amount of between 0.2 mm and 0.5 mm, while the height of cutting conductor 36 can have an amount of between 0.4 mm and 1.00 mm. The coating can consist of copper, aluminum or silver, for example. Copper comprises a specific electrical resistance of approximately 0.01 Ω·mm²/m and a thermal conductivity between 240 and 380 W/m·K. Silver and aluminum comprise, however, a specific electrical resistance of approximately 0.016 Ω·mm²/m and 0.026 Ω·mm²/m as well as a thermal conductivity of approximately 429 W/m·K and 160 W/m·K. The cutting conductor 36 and/or the return conductor 37 consist preferably of stainless steel, Inconel or Kanthal-D, having a comparably high specific resistance of approximately between 0.7 and 1.5 Ω·mm²/m and a comparably low thermal conductivity between 15 and 25 W/m·K.

The insulating body 34 is thermally and electrically insulating. This results in that the heat in the central area between the jaws 15, 16 is mainly maintained only on the cutting conductor 36. On the contrary, heat that is transferred in the distal end section 28 of first jaw 15 due to heat conduction into the return conductor 37 is particularly distributed by means of coating 43 of return conductor 37 and can be extensively dissipated without creating local hotspots. This allows to configure the jaws 15, 16 particularly slim, thin and flat without heating the outer sides of the jaws, particularly during long operation.

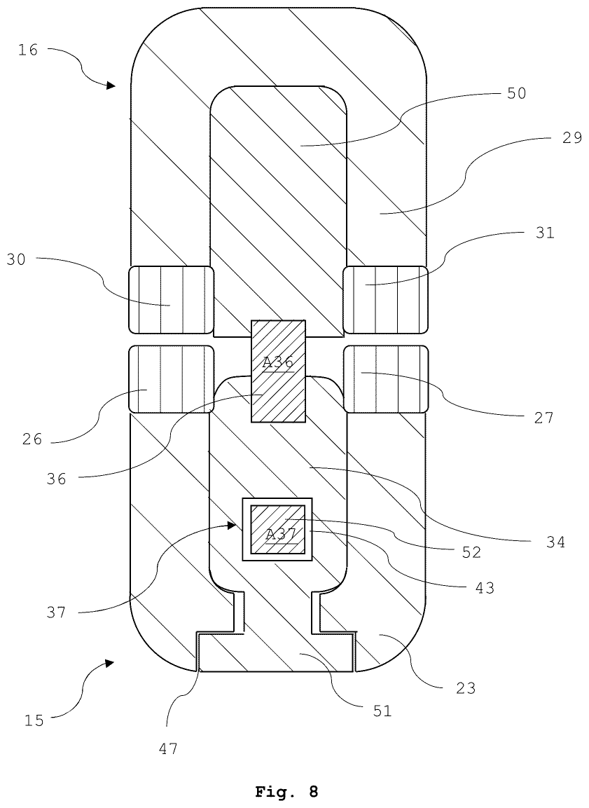

Different to the illustration in FIG. 7, the support part 23 of first jaw 15 can comprise pockets 47 at its lower side around opening 46, so that the insulating body feet 51 can be cut away flush with support part 23. This is illustrated in FIG. 8.

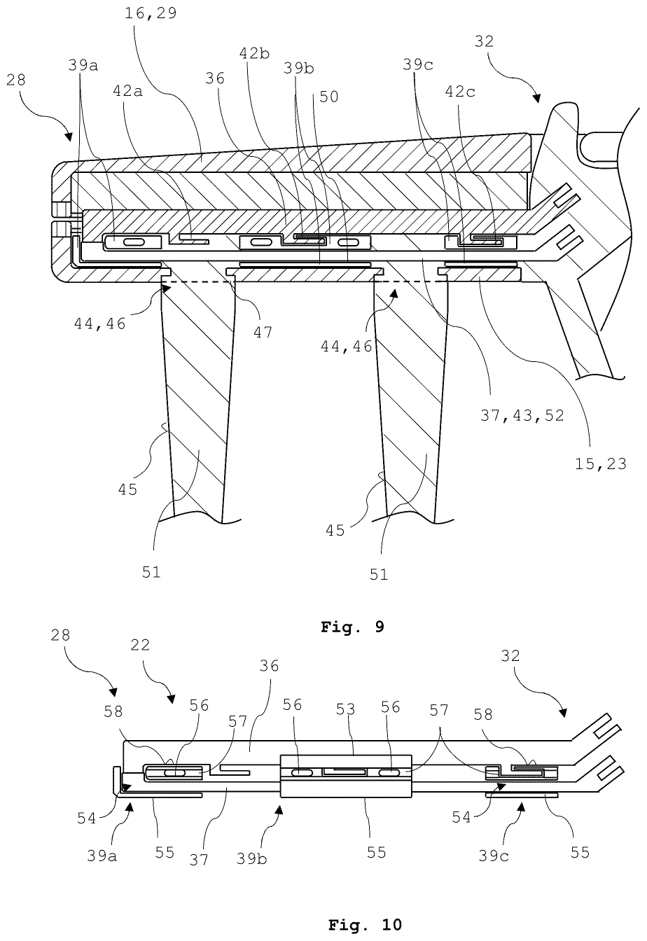

In FIGS. 9 to 12 another example for the configuration of the jaws is illustrated. For the example depicted in FIGS. 9 to 12 the above explanation applies accordingly with reference to the reference signs.

This example distinguishes from the preceding examples in that the first jaw 15 comprises only two insulating body feet 51, but three distance elements 39a, 39b, 39c. The cutting conductor 36 comprises different undercut sections 42a, 42b, 42c that can be either positioned inside insulating body 34 (compare undercut section 42a) or inside a distance element 39b, 39c (compare undercut sections 42b, 42c).

Together with the undercut sections 42b, 42c the distance elements 39a, 39b, 39c form a form-fit and fixate the cutting conductor 36, whereby lifting of the cutting conductor 36 due to tissue sticking can be avoided. The distance elements 39a, 39b, 39c avoid the electrical contact between cutting conductor 36 and return conductor 37 as well as to the support part 23 of first jaw 15.

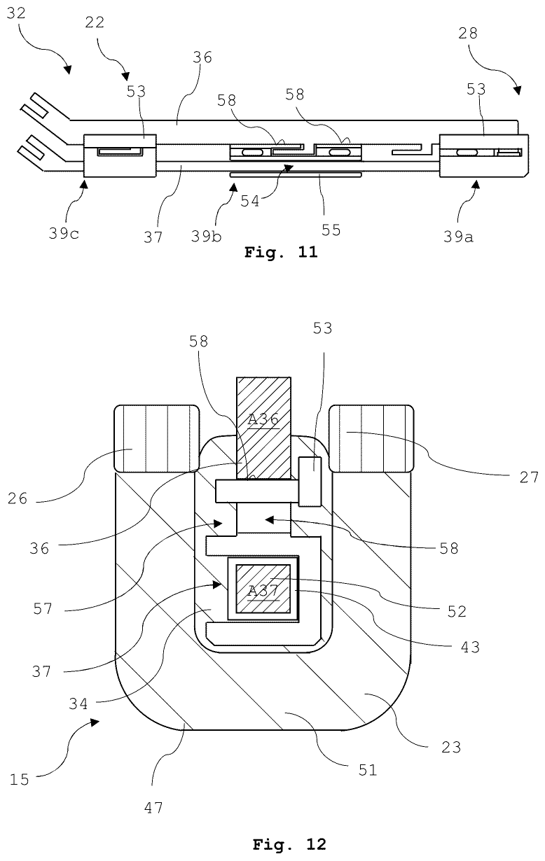

As illustrated in FIG. 10 from one side and in FIG. 11 from the other side, the distance elements 39a, 39b, 39c comprise additionally lateral stops 53 by means of which the centered positioning of the cutting element 22 in first jaw 15 can be guaranteed. The lateral stops 53 laterally adjoin a support surface 58 on which cutting conductor 36, particularly its lower side, is positioned and can be supported. The distance elements 39a, 39b, 39c comprise return conductor cavities 54 that are respectively open to one lateral side. The return conductor cavities 54 can be alternatingly plugged on the cutting conductor 36 and return conductor 37 whereby a centered positioning in the support part 23 of first jaw 15 can be achieved. The proximal distance element 39c comprises in addition a bottom plate 55 adapted to the support part 23, wherein the bottom plate 55 tapers in direction toward the distal end 28 of first jaw 15, so that the cutting conductor 36 can also be positioned centrally in the enlarging proximal area 32 close to the joint.

Additionally, the distance elements 39a, 39b, 39c comprise transverse as well as longitudinal cavities 56, 57 through which the material of insulating body 34 can flow during production. A form-fit between the distance elements 39a, 39b, 39c and the insulating body 34 can be created in this manner.

The combination of the different form-fits—also the form-fit between the undercut sections 44 of insulating body 51—can prevent lifting of the cutting element 22.

The material of the distance elements 39a, 39b, 39c can be ceramic with electrically and thermally insulating characteristics, whereby it can be avoided that the produced heat is distributed in the cutting element 22, but is instead mainly output to the tissue at the foreseen position—the cutting conductor 36.

The invention refers to an electrosurgical instrument 10, particularly for thermal cutting of biological tissue of a patient. The instrument 10 comprises two jaws 15, 16, wherein at least one of the jaws 15 is configured to be moved relative to the other jaw 16 toward and away from the latter. At least one of the jaws 15 comprises a thermal cutting element 22, having an electrically conductive cutting conductor 36 and an electrically conductive return conductor 37 that are electrically connected with each other. The cutting element 22 is arranged, so that the cutting conductor 36 is at least thermally uncovered on a side of one jaw 15 facing the other jaw 16 and so that the return conductor 37 is surrounded by an insulating body 34. The return conductor 37 comprises a lower electrical resistance in current flow direction and preferably a higher thermal conductivity than the cutting conductor 36. In doing so, during supply of current to the cutting element 22, at the return conductor 37 a considerably lower amount of heat and temperature is created than at the cutting conductor 36 and the return conductor 37 can be used for heat dissipation.

List of Reference Signs:

| | |
|---|---|
| 10 | electrosurgical instrument |
| 11 | shank |
| 12 | proximal end of the instrument |
| 13 | distal end of the instrument |
| 14 | tool |
| 15 | first jaw |
| 16 | second jaw |
| 17 | handle |
| 18 | electrical supply line |
| 19 | instrument supply line |
| 20 | supply apparatus |
| 21 | pivot axis |
| 22 | cutting element |
| 23 | support part of first jaw |
| 24 | outer side of support part |
| 25 | edge regions of the jaws |
| 26, 27 | sealing electrode of first jaw |
| 28 | distal end section of the jaws |
| 29 | support part of second jaw |
| 30, 31 | sealing electrode of second jaw |
| 32 | area close to the joint |
| 33 | groove of first jaw |
| 34 | insulation body |
| 35 | groove of second jaw |
| 36 | cutting conductor |
| 37 | return conductor |
| 38 | connection elements |
| 39 | distance element |
| 40 | positioning projections |
| 41 | attachment projections |
| 42 | undercut sections |
| 43 | coating |
| 44 | undercut section of insulation body feet |
| 45 | tapering section of insulation body feet |
| 46 | openings in support part of first jaw |
| 47 | pockets |
| 48 | RF generator |
| 49 | transformer |
| 50 | counter pressing body |
| 51 | insulation body feet |
| 52 | core of return conductor |
| 53 | lateral stop |
| 54 | return conductor cavity |
| 55 | bottom plate |
| 56 | transverse cavity |
| 57 | longitudinal cavity |
| 58 | support surface |
| A36 | cross section area of cutting conductor |
| A37 | cross section area of return conductor |

1. An electrosurgical instrument (10) for thermal cutting of biological tissue comprising:
at least two jaws (15, 16), wherein at least one of the at least two jaws (15, 16) is a movable jaw that is movable toward and away from another of the at least two jaws (16, 15), wherein at least one of the two jaws (15, 16) comprises a thermal cutting element (22) having an electrically conductive cutting conductor (36) and an electrically conductive return conductor (37) connected with the cutting conductor (36);
wherein the thermal cutting element (22) is arranged, so that the cutting conductor (36) is at least partly thermally uncovered on a side of the at least one of the at least two jaws (15) facing the other of the at least two jaws (16) and so that the return conductor (37) is surrounded by an insulating body (34); and
wherein the return conductor (37) comprises a lower electrical resistance than the cutting conductor (36).

2. The electrosurgical instrument (10) according to claim 1, wherein the return conductor (37) has a higher thermal conductivity than the cutting conductor (36).

3. The electrosurgical instrument (10) according to claim 1, wherein the cutting conductor (36) and the return conductor (37) are of one monolithic body configured in a U-shaped manner.

4. The electrosurgical instrument (10) according to claim 3, wherein the cutting conductor (36) and the return conductor (37) are thermally and electrically connected with each other in a distal end section (28) of the at least one of the at least two jaws (15).

5. The electrosurgical instrument (10) according to claim 1, wherein a core (52) of the return conductor (37) is at least partly provided with a coating (43), wherein the coating (43) comprises a material that has a lower specific electrical resistance and/or a higher specific thermal conductivity than a base material of the cutting element (22).

6. The electrosurgical instrument (10) according to claim 1, wherein the cutting conductor (36) has a larger cross-sectional area than the return conductor (37).

7. The electrosurgical instrument (10) according to claim 1, wherein the at least one of the at least two jaws (15, 16) comprises at least one sealing electrode (26, 27) in an edge region (25) thereof, wherein the at least one sealing electrode (26, 27) is distanced from the cutting conductor (36), wherein at lateral flanks of the cutting conductor (36), the cutting conductor is surrounded by the sealing electrode (26, 27) between distal and proximal ends of the at least one of the at least two jaws (15) and/or at a distal end section (28) of the at least one of the at least two jaws (15).

8. The electrosurgical instrument (10) according to claim 7, wherein the other of the at least two jaws (16) comprises at least one sealing electrode (30, 31) in an edge region (25) thereof, wherein the at least one sealing electrode (30, 31) is arranged identically to the sealing electrode (26, 27) of the at least one of the at least two jaws (15).

9. The electrosurgical instrument (10) according to claim 8, wherein the sealing electrodes (26, 27; 30, 31) of the at least two jaws are arranged opposite one another and are distanced from each other when the at least two jaws (15, 16) are closed.

10. The electrosurgical instrument (10) according to claim 1, wherein the insulating body (34) comprises a material that is electrically insulating and has a lower thermal conductivity than the cutting element (22).

11. The electrosurgical instrument (10) according to claim 1, wherein the cutting conductor (36) comprises at least one attachment projection (41), wherein the at least one attachment projection (41) is surrounded by the insulating body (34), wherein the at least one attachment projection (41) comprises an undercut section (42) which is enclosed by the insulating body (34).

12. The electrosurgical instrument (10) according to claim 1, wherein at least one distance element (39, 39a, 39b, 39c) is arranged between the cutting conductor (36) and the return conductor (37), wherein the at least one distance element comprises a material that is electrically and thermally insulating.

13. The electrosurgical instrument according to claim 12, wherein the at least one distance element (39) comprises an upper side and a lower side, wherein the at least one distance element (39) is in contact with the cutting conductor (36) on the upper side of the at least one distance element and/or is in contact with the return conductor (37) on the lower side of the at least one distance element.

14. The electrosurgical instrument (10) according to claim 12, wherein the at least one distance element (39a, 39b, 39c) comprises a return conductor cavity (54) in which the return conductor (37) is arranged, such that the return conductor is at least partly enclosed by and is held by the at least one distance element (39a, 39b, 39c).

15. The electrosurgical instrument (10) according to claim 14, wherein the at least one distance element (39a, 39b, 39c) comprises a support surface (58) on which the cutting conductor (36) is disposed, wherein the distance element (39a) comprises at least one lateral stop (53) that projects from the support surface (58) and against which the cutting conductor abuts laterally.

16. The electrosurgical instrument (10) according to claim 1, wherein the other jaw of the at least two jaws (16) comprises a counter-pressing body (50) which is arranged on a side facing the at least one of the at least two jaws (15) and which comprises a counter-pressing surface for the cutting conductor 36 of the at least one of the at least two jaws (15).

17. The electrosurgical instrument (10) according to claim 1, wherein the cutting conductor (36) projects from a surface formed by the insulating body (34) toward the other jaw of the at least two jaws (16).

18. The electrosurgical instrument (10) according to claim 16, wherein the counter-pressing body (50) and the cutting conductor (36) overlap along the cutting conductor (36) with a uniform depth when the at least two jaws (15, 16) are closed, wherein the cutting conductor (36) deforms the counter-pressing body (50).
